(19) United States
(12) Patent Application Publication
(10) Pub. No.: US 2018/0028351 A1
(43) Pub. Date: Feb. 1, 2018

(54) ANTI-SNORE DEVICES

(71) Applicant: Quanta Computer Inc., Taoyuan City (TW)

(72) Inventors: Chia-Yuan CHANG, Taoyuan City (TW); Ya-Chyi CHOU, Taoyuan City (TW); Kao-Yu HSU, Taoyuan City (TW)

(21) Appl. No.: 15/371,529

(22) Filed: Dec. 7, 2016

(30) Foreign Application Priority Data
Jul. 26, 2016 (TW) 105211238

(51) Int. Cl. A61F 5/56; A47G 9/10; A61B 5/145; A61B 5/00; A61B 7/00

(52) U.S. Cl. CPC A61F 5/56; A61B 5/4806; A61B 5/4836; A61B 7/003; A61B 5/14542; A47G 9/1081; A47G 9/1027; A47G 2009/1018

(57) ABSTRACT

An anti-snore device includes a headrest, a snoring detector, an inflator, and a pressure detector. The headrest includes a first airbag and a second airbag. The snoring detector detects snore information to transmit an enable signal. The inflator inflates the first airbag or the second airbag according to the enable signal and deflates the first airbag or the second airbag according to a disable signal. The pressure detector is configured to detect the pressure in the first airbag or the second airbag. When the pressure detector has detected that the pressure in the first airbag or the second airbag exceeds a predetermined pressure value, the pressure detector transmits the disable signal.

### CROSS REFERENCE TO RELATED APPLICATIONS

This application claims priority of Taiwan Patent Application No. 105211238, filed on Jul. 26, 2016, the entirety of which is incorporated by reference herein.

### BACKGROUND OF THE INVENTION

### Field of the Invention

The disclosure relates generally to anti-snore devices, and more particularly it relates to headsets configured to stop users from snoring.

### Description of the Related Art

A good headset can not only stabilize the wearer's sleeping posture but also reduce compression in the cervical vertebrae to provide a good-quality sleep. Since people spend about one-third of life time on lying on bed, it should be very happy to find a sleeping posture, which is suitable for their own, and a good headset for the sake of falling asleep easily. A person's seep quality may suffer due to snoring, and the reason for snoring is mainly airway obstruction, resulting in reduced sleep quality.

In addition, some users use headsets that they are familiar with as they sleep. Once they sleep without their headsets that they are familiar with, they turn to be really hard to fall asleep. Therefore, if the users may improve their snoring phenomenon with their headsets that they are familiar with, the users could have an excellent sleeping quality to improve their daily working efficiency.

### BRIEF SUMMARY OF THE INVENTION

In an embodiment, an anti-snore device comprises a headset, a snoring detector, an inflator, and a pressure detector. The headset comprises a first airbag and a second airbag. The snoring detector detects snore information to transmit an enable signal. The inflator inflates either the first airbag or the second airbag according to the enable signal. The inflator deflates the first airbag or second airbag according to a disable signal. The pressure detector is configured to detect pressure in the first airbag or the second airbag. The pressure detector transmits the disable signal when the pressure detector has detected that the pressure in the first airbag or second airbag exceeds a predetermined pressure value.

According to an embodiment of the invention, the headset further comprises: a first tube and a second tube. The first tube is coupled between the first airbag and the inflator, such that the inflator inflates and deflates the first airbag through the first tube. The second tube is coupled between the second airbag and the inflator, such that the inflator inflates and deflates the second airbag through the second tube. The pressure detector detects pressure in the first tube and in the second tube respectively to determine whether the pressure of the first airbag or the second airbag exceeds the predetermined pressure value.

According to an embodiment of the invention, the headset further comprises a hard foam and a soft foam. The hard foam comprises a plurality of slots. The first tube and the second tube are deposited in the slots. The soft foam is deposited on the hard foam. The first airbag and the second airbag are deposited side-by-side at the bottom of the headset, the hard foam is deposited on the first airbag and the second airbag, and the hard foam is deposited between the soft foam and both the first airbag and the second airbag.

According to an embodiment of the invention, the anti-snore device further comprises a headset cover. The headset cover is configured to cover the headset.

According to an embodiment of the invention, the snoring detector is a microphone configured to detect a snoring sound to generate the enable signal. The microphone is deposited at the top of the headset.

According to an embodiment of the invention, the snoring detector is an Oxymeter configured to detect the blood oxygen concentration of a user to transmit the enable signal.

According to an embodiment of the invention, when the snoring detector has detected the snore information once again, the inflator inflates the other of the first airbag and the second airbag. When the pressure detector has detected that the pressure of the other of the first airbag and the second airbag exceeds the predetermined pressure value, the inflator deflates the other of the first airbag and the second airbag.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1 is a block diagram of an anti-snore device in accordance with an embodiment of the invention;

FIG. 2 is a schematic diagram of an anti-snore device in accordance with an embodiment of the invention;

FIGS. 3A and 3B are operation diagrams of an anti-snore device in accordance with an embodiment of the invention; and

FIG. 4 is a cross-section diagram of a headset in accordance with an embodiment of the invention.

### DETAILED DESCRIPTION OF THE INVENTION

This description is made for the purpose of illustrating the general principles of the invention and should not be taken in a limiting sense. In addition, the present disclosure may repeat reference numerals and/or letters in the various examples. This repetition is for the purpose of simplicity and clarity and does not in itself dictate a relationship between the various embodiments and/or configurations discussed. The scope of the invention is best determined by reference to the appended claims.

It should be understood that the following disclosure provides many different embodiments, or examples, for implementing different features of the application. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Moreover, the formation of a feature on, connected to, and/or coupled to another feature in the present disclosure that follows may include embodiments in which the features are formed in direct contact, and may also include embodiments in which additional features may be formed interposing the features, such that the features may not be in direct contact.

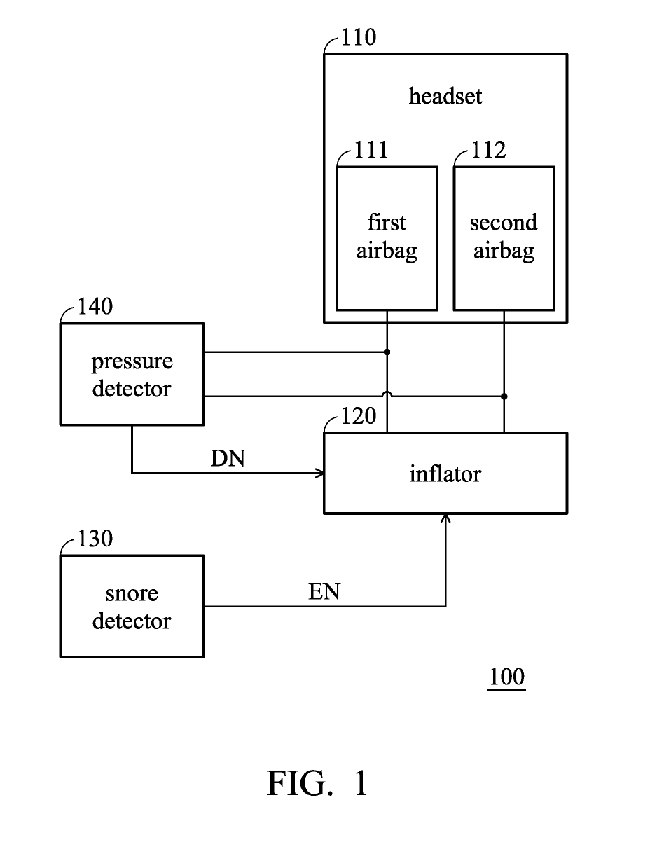

FIG. 1 is a block diagram of an anti-snore device in accordance with an embodiment of the invention. As shown in FIG. 1, the anti-snore device 100 includes a headset 110, an inflator 120, a snore detector 130, and a pressure detector 140. The headset 110 includes a first airbag 111 and a second airbag 112. The inflator 120 inflates the first airbag 111 or the second airbag 112 according to the enable signal EN, and deflates the first airbag 111 or the second airbag 112 according to the disable signal DN. According to other embodiments of the invention, the headset 110 may include a plurality of airbags. The first airbag 111 and the second airbag 112 are merely illustrated herein.

The snoring detector 130 detects the snore information to transmit the enable signal EN to the inflator 120. The pressure detector 140 is configured to detect the pressure of the first airbag 111 or the second airbag 112. When the pressure detector 140 detects that the pressure of the first airbag 111 or the second airbag 112 exceeds a predetermined pressure value, the pressure detector 140 generates the disable signal DN.

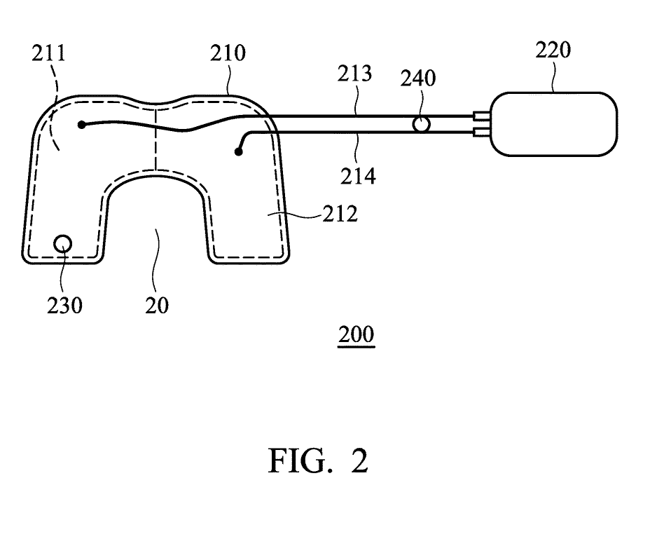

FIG. 2 is a schematic diagram of an anti-snore device in accordance with an embodiment of the invention. As shown in FIG. 2, the headset 210 includes the first airbag 211, the second airbag 212, the first tube 213, and the second tube 214, in which the first airbag 211 and the second airbag 212 is deposited side-by-side at the bottom of the headset 210, the first tube 213 is coupled to the first airbag 211, and the second tube 214 is coupled to the second airbag 212.

The headset 210, the first airbag 211, the second airbag 212, the inflator 220, the snoring detector 230, and the pressure detector 240 shown in FIG. 2 respectively correspond to the headset 110, the first airbag 111, the second airbag 112, the inflator 120, the snoring detector 130, and the pressure detector 140 shown in FIG. 1.

The inflator 220 inflates, according to the enable signal EN (as shown in FIG. 1), the first airbag 211 through the first tube 213 and inflates the second airbag 212 through the second tube 214. In addition, the inflator 220 deflates, according to the disable signal (as shown in FIG. 1), the first airbag 211 through the first tube 213 and deflates the second airbag 212 through the second tube 214.

The snoring detector 230 is configured to collect the snore information and transmits the enable signal EN (as shown in FIG. 1) to the inflator 220. According to an embodiment of the invention, the snoring detector 230 is a microphone located at the top of the headset 210, configured to detect the snoring of the user to transmit the enable signal EN (as shown in FIG. 1) to the inflator 220. The microphone is as close to the user as possible, in order to precisely detect the snoring of the user.

According to another embodiment of the invention, the snoring detector 230 is an Oxymeter wearing on the user (i.e., separated from the headset 210), which is configured to detect the user's blood oxygen concentration. When the Oxymeter detects the blood oxygen concentration has decreased to a threshold, the Oxymeter determines that the user is experiencing sleep apnea and transmits the enable signal EN (as shown in FIG. 1) to the inflator 220, such that the inflator 220 inflates the first airbag 211 or the second airbag 212.

The pressure detector 240 detects the pressure of the first airbag 211 and the second airbag 212 through the first tube 213 and the second tube 214 respectively to determine whether the pressure of the first airbag 211 or the second airbag 212 exceeds the predetermined pressure value. According to an embodiment of the invention, when the pressure detector 240 determines, through the first tube 213, that the pressure of the first airbag 211 exceeds the predetermined pressure value, the pressure detector 240 generates the disable signal DN (as shown in FIG. 1), such that the inflator 220 deflates the first airbag 211. According to another embodiment of the invention, when the pressure detector 240 determines, through the second tube 214, that the pressure of the second airbag 212 exceeds the predetermined pressure value, the pressure detector 240 generates the disable signal DN (as shown in FIG. 1), such that the inflator 220 deflates the second airbag 212.

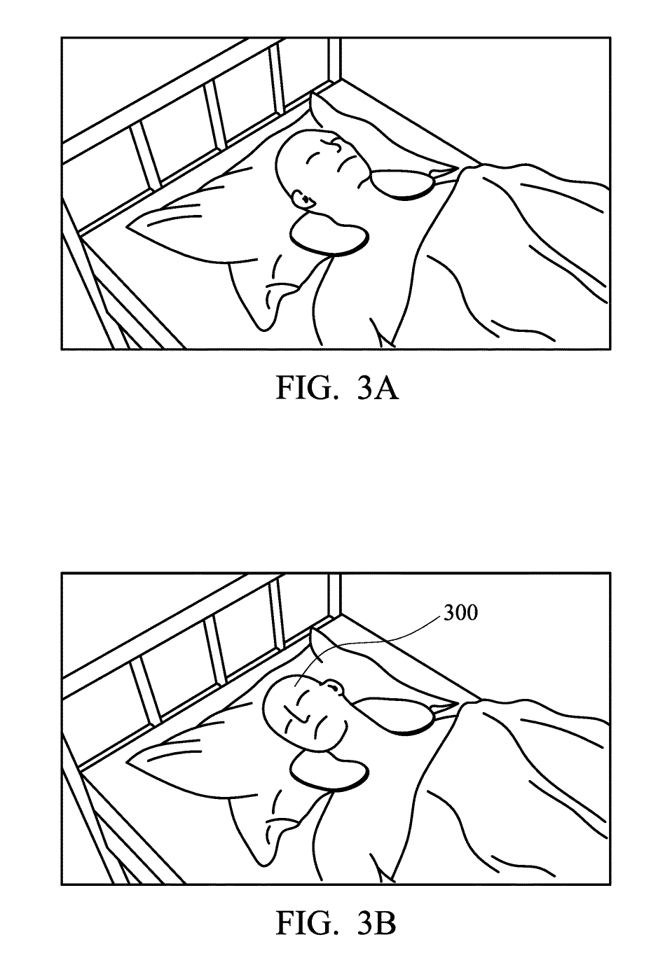

As shown in FIG. 2, the first airbag 211 and the second airbag 212 are deposited side-by-side in both sides of the headset 210. FIGS. 3A and 3B are operation diagrams of an anti-snore device in accordance with an embodiment of the invention. In FIGS. 3A and 3B, the head of the user 300 is lying on the headset 310, in which headset 310 corresponds to the headset 210 in FIG. 2 and the neck of the user 300 is deposited in the slot 20 in FIG. 2.

According to an embodiment of the invention, when the snoring detector 230 has detected the snore information to transmit the enable signal EN, the inflator 220 inflates the first airbag 211, such that the head of the user 300 is rotated in the direction shown from FIG. 3A to FIG. 3B (i.e., rotated toward the direction of the second airbag 212). When the pressure detector 240 has detected that the pressure of the first airbag 211 exceeds the predetermined pressure value to generate the disable signal DN, the inflator 220 immediately deflates the first airbag 211. At this moment, the head of the user 300 is rotated due to the inflated airbag 211.

According to another embodiment of the invention, when the snoring detector 230 has detected the snore information generated by the user 300 once again to generate the enable signal EN, the inflator 220 inflates the second airbag 212, such that the head of the user 300 is rotated in the opposite direction shown in FIG. 3B (i.e., rotated toward the second airbag 212). When the pressure detector 240 has detected that the pressure of the second airbag 212 exceeds the predetermined pressure value to generate the disable signal DN, the inflator 220 immediately deflates the second airbag 212. At this moment, the head of the user 300 is rotated due to the inflated second airbag 212.

According to other embodiments of the invention, whenever the snoring detector 230 detects the snore information generated by the user 300 to transmit the enable signal EN, the inflator 220 alternatively inflates the first airbag 211 and the second airbag 212 to rotate the head of the user 300. In addition, when the pressure detector 240 detects that the pressure of the second airbag 212 exceeds the predetermined pressure value, the pressure detector 240 generates the disable signal DN, such that the inflator 220 deflates the first airbag 211 or the second airbag 212 that has been inflated.

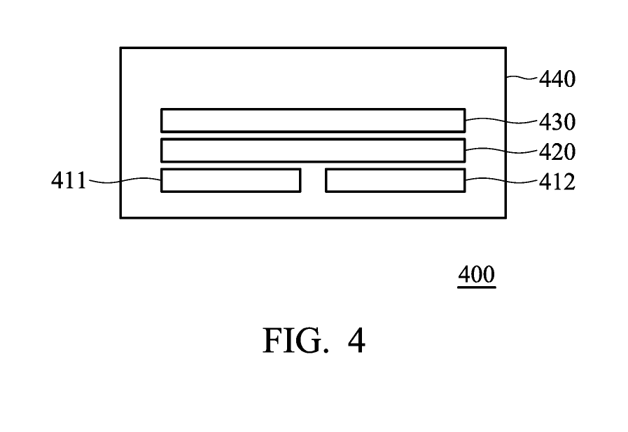

FIG. 4 is a cross-section diagram of a headset in accordance with an embodiment of the invention. As shown in FIG. 4, the headset 400 includes a first airbag 411, a second airbag 412, a hard foam 420, a soft foam 430, and a headset cover 440, in which the first airbag 411 and the second airbag 412 are deposited side-by-side at the bottom of the headset 400.

The hard foam 420 includes a plurality of slots (not shown in FIG. 4). The first tube 213 and the second tube 214 shown in FIG. 2 are deposited in the slots for preventing the first tube 213 and the second tube 214 from being squeezed, resulting in impeding the first airbag 211 and the second airbag 212 from being inflated or deflated. The soft foam 430 is deposited on the hard foam 420 as a shock absorber.

The invention allows the user to sleep with his/her familiar headset with the use of the anti-snore device provided herein, without changing to a new headset with special specifications. The user only has to lie on the headset as usual to operate the anti-snore device. When the snoring detector detects that the user is snoring, the inflator then individually inflates the airbags to rotate the user's head, so that the respiratory tract is unblocked.

When the pressure in a tube has reached the predetermined pressure value, the corresponding airbag immediately stops being inflated and begins to be deflated. After the airbag has been deflated, the other airbag will be inflated again when the snoring detector detects that the user is snoring again, and the cycle is repeated to achieve the purpose of stopping the snoring. The tube can be disassembled for storage, the air in the airbags can be emptied for the convenience of storage and carrying, and the headset cover can be removed for cleaning and changing.

While the invention has been described by way of example and in terms of preferred embodiment, it is to be understood that the invention is not limited thereto. Those who are skilled in this technology can still make various alterations and modifications without departing from the scope and spirit of this invention. Therefore, the scope of the present invention shall be defined and protected by the following claims and their equivalents.

What is claimed is:

1. An anti-snore device, comprising:
a headset, comprising:
a first airbag; and
a second airbag;
a snoring detector, detecting snore information to transmit an enable signal;
an inflator, inflating the first airbag or the second airbag according to an enable signal and deflating the first airbag or second airbag according to a disable signal; and
a pressure detector, configured to detect pressure in the first airbag or second airbag, wherein the pressure detector transmits the disable signal when the pressure detector has detected that the pressure in the first airbag or second airbag exceeds a predetermined pressure value.

2. The anti-snore device of claim 1, wherein the headset further comprises:
a first tube, coupled between the first airbag and the inflator, such that the inflator inflates and deflates the first airbag through the first tube; and
a second tube, coupled between the second airbag and the inflator, such that the inflator inflates and deflates the second airbag through the second tube, wherein the pressure detector detects pressure in the first tube and in the second tube respectively to determine whether the pressure of the first airbag or the second airbag exceeds the predetermined pressure value.

3. The anti-snore device of claim 2, wherein the headset further comprises:
a hard foam, comprising a plurality of slots, wherein the first tube and the second tube are deposited in the slots; and
a soft foam, deposited on the hard foam, wherein the first airbag and the second airbag are deposited side-by-side at the bottom of the headset, the hard foam is deposited on the first airbag and the second airbag, and the hard foam is deposited between the soft foam and both the first airbag and the second airbag.

4. The anti-snore device of claim 3, further comprising:
a headset cover, wherein the headset cover is configured to cover the headset.

5. The anti-snore device of claim 1, wherein the snoring detector is a microphone configured to detect a snoring sound to generate the enable signal, wherein the microphone is deposited at the top of the headset.

6. The anti-snore device of claim 1, wherein the snoring detector is an Oxymeter configured to detect the blood oxygen concentration of a user to transmit the enable signal.

7. The anti-snore device of claim 1, wherein, when the snoring detector has detected the snore information once again, the inflator inflates the other of the first airbag and the second airbag, wherein, when the pressure detector has detected that the pressure of the other of the first airbag and the second airbag exceeds the predetermined pressure value, the inflator deflates the other of the first airbag and the second airbag.
